(19) United States
(12) Patent Application Publication
(10) Pub. No.: US 2009/0054439 A1
(43) Pub. Date: Feb. 26, 2009

(54) SULFONAMIDE DERIVATIVES, PREPARATION AND THERAPEUTIC APPLICATION THEREOF

(75) Inventors: Gilles COURTEMANCHE, Antony (FR); Pierre DESPEYROUX, Antony (FR); Evelyne FONTAINE, Antony (FR); Pierrick ROCHARD, Antony (FR); Claudine SERRADEIL-LE GAL, Antony (FR); Erich Von Roedern, Frankfurt (DE)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/183,487

(22) Filed: Jul. 31, 2008

Related U.S. Application Data
(63) Continuation of application No. PCT/FR2007/000182, filed on Feb. 1, 2007.

(30) Foreign Application Priority Data
Feb. 2, 2006 (FR) 0600955

(51) Int. Cl. A61K 31/5375; C07C 311/29; C07D 265/30; A61K 31/18

(52) U.S. Cl. 514/238.2; 564/89; 514/604; 544/159

(57) ABSTRACT

The present invention is directed to a compound of formula (I):

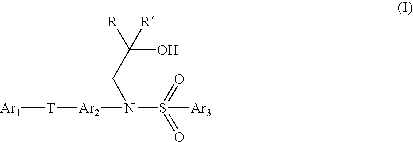

wherein Ar₁, Ar₂, Ar₃, R, R′ and T are as defined herein, its preparation, pharmaceutical composition and uses as orexin 2 receptor antagonist.

This application is a Continuation of International Application No. PCT/FR2007/000182, filed Feb. 1, 2007, which is incorporated by reference herein in its entirety.

### FIELD OF THE INVENTION

The subject matter of the present invention is sulfonamide derivatives, their process of preparation and their therapeutic use.

### BACKGROUND OF THE INVENTION

Orexins A and B (or hypocretins 1 and 2) are hypo-thalamic neuropeptides of 33 and 28 amino acids respectively, recently identified as endogenous ligands of two seven-domain transmembrane receptors, named orexin 1 and orexin 2 receptors (Sakurai T., Cell, Vol. 92, 573-585, 1998; De Lecea L., Proc. Natl. Acad. Sci., Vol. 95, 322-327, 1998).

The orexin 2 receptor has the property of recognizing the two forms of orexin A and B equivalently. In contrast, the orexin 1 receptor, which has 64% homology with the orexin 2 receptor, is more selective and binds orexin A ten times better than orexin B (Sakurai T., Cell, Vol. 92, 573-585, 1998).

The orexins control various central and peripheral functions via these receptors, in particular intake of food and drink, certain cardiovascular endocrine functions and the wake/sleep cycle (Sakurai T., Regulatory Peptides, Vol. 85, 25-30, 1999).

It has now been found that some sulfonamide derivatives exhibit a high affinity with regard to the orexin 2 receptors and are powerful antagonists of these receptors.

### SUMMARY OF THE INVENTION

Thus, a subject matter of the present invention is compounds corresponding to the general formula (I):

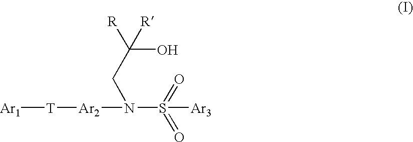

in which:
R represents:
   a hydrogen atom;
   a (C₁-C₄)alkyl;
R′ represents:
   a (C₁-C₄)alkyl optionally substituted by one or more groups chosen from:
      a hydroxyl group,
      a halogen group,
      a (C₁-C₄)alkoxy group,
      an aryloxy group, it being possible for said aryl to be substituted by one or more groups chosen from: a halogen atom, a (C₁-C₄)alkyl or a (C₁-C₄)alkoxy;
      an aryl group, it being possible for said aryl to be substituted by one or more groups chosen from: a halogen atom, a (C₁-C₄)alkyl or a (C₁-C₄) alkoxy;
      an —NH(COO)Rₐ group, in which Rₐ represents a (C₁-C₄) alkyl;
      an NH(CO)R_{b} group, in which R_{b} represents a (C₁-C₄)alkyl or an aryl group, said aryl group optionally being substituted by one or more groups chosen from: a halogen atom, a (C₁-C₄)alkyl, a (C₁-C₄)alkoxy or a carboxylic acid;
      an —NR_{c}R_{d} group, in which R_{c} and R_{d} represent, independently of one another: a hydrogen atom or a (C₁-C₄)alkyl, or form, with the nitrogen atom which connects them, a heterocyclyl;
      an —NR_{f}R_{g}Rₕ ammonium group, in which R_{f}, R_{g} and Rₕ represent a (C₁-C₄)alkyl;
      a heterocyclyl group;
      a COORₑ group, in which Rₑ can represent a hydrogen or a (C₁-C₄)alkyl;
      a CONR_{c}R_{d} group;
   a COOH, a COO(C₁-C₄)alkyl or a CONR_{c}R_{d};
Ar₁ represents:
an aryl group optionally substituted by one or more groups chosen, independently of one another, from the following groups: a halogen atom, a cyano, a (C₁-C₄)alkyl, a fluoro(C₁-C₄)alkyl or a (C₁-C₄)alkoxy;
a heterocyclyl group optionally substituted by a halogen atom, a (C₁-C₄)alkyl or a (C₁-C₄)alkoxy;
T represents:
a —(CH₂)ₙ— group with n=1 or 2;
a group:

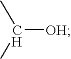

Ar₂ represents:
an aryl group optionally substituted by one or more groups chosen, independently of one another, from the following groups: a halogen atom, a (C₁-C₄)alkyl, a (C₁-C₄)alkoxy, a fluoro(C₁-C₄)alkyl or a fluoro(C₁-C₄)alkoxy;
a heterocyclyl group optionally substituted by a halogen atom, a (C₁-C₄)alkyl or a (C₁-C₄)alkoxy;
Ar₃ represents:
an aryl group optionally substituted by one or more groups chosen, independently of one another, from the following groups: a halogen atom, a hydroxyl group, a (C₁-C₄)alkyl or a (C₁-C₄)alkoxy;
a heterocyclyl group optionally substituted by a hydroxyl group, a (C₁-C₄)alkyl, a (C₁-C₄)alkoxy, a fluoro(C₁-C₄)alkyl or a fluoro(C₁-C₄)alkoxy; in the base, addition salt with an acid, hydrate or solvate state, in the form of enantiomers, of diastereoisomers, of rotamers, of atropoisomers or of their mixtures.

### DETAILED DESCRIPTION OF THE INVENTION

Mention may be made, among the compounds which are subject matters of the invention, of a second group of compounds of general formula (I) in which:
R represents:
   a hydrogen atom;
   a (C₁-C₄)alkyl;
R′ represents:
   a (C₁-C₄)alkyl optionally substituted by one or more groups chosen from:
      a hydroxyl group,
      a halogen group,
      a (C₁-C₄)alkoxy group,
      an aryloxy group, it being possible for said aryl to be substituted by one or more groups chosen from: a halogen atom, a (C₁-C₄)alkyl or a (C₁-C₄) alkoxy;
      an aryl group, it being possible for said aryl to be substituted by one or more groups chosen from: a halogen atom, a (C₁-C₄)alkyl or a (C₁-C₄) alkoxy;
      an —NH(COO)Rₐ group, in which Rₐ represents a (C₁-C₄) alkyl;
      an NH(CO)R_{b} group, in which R_{b} represents an aryl group, said aryl group optionally being substituted by one or more groups chosen from: a halogen atom, a (C₁-C₄)alkyl, a (C₁-C₄)alkoxy or a carboxylic acid;
      an —NR_{c}R_{d} group, in which R_{c} and R_{d} represent, independently of one another: a hydrogen atom or a (C₁-C₄)alkyl, or form, with the nitrogen atom which connects them, a heterocyclyl;
      an —NR_{f}R_{g}Rₕ ammonium group, in which R_{f}, R_{g} and Rₕ represent a (C₁-C₄) alkyl;
      a heterocyclyl group;
      a COORₑ group, in which Rₑ can represent a hydrogen or a (C₁-C₄)alkyl;
      a CONR_{c}R_{d} group;
   a COO(C₁-C₄)alkyl or a CONR_{c}R_{d};
Ar₁ represents:
a phenyl or a naphthyl optionally substituted by one or more groups chosen, independently of one another, from the following groups: a halogen atom, a (C₁-C₄)alkyl or a (C₁-C₄)alkoxy;
a heterocyclyl group, in particular pyridinyl or pyrimidinyl, said heterocyclyl group optionally being substituted by a halogen atom, a (C₁-C₄)alkyl or a (C₁-C₄) alkoxy;
T represents:
a —CH₂— group;
Ar₂ represents:
a phenyl or a naphthyl optionally substituted by one or more groups chosen, independently of one another, from the following groups: a halogen atom, a (C₁-C₄)alkyl or a (C₁-C₄)alkoxy;
a heterocyclyl group, in particular pyridinyl, optionally substituted by a halogen atom, a (C₁-C₄)alkyl or a (C₁-C₄)alkoxy;
Ar₃ represents
a phenyl or a naphthyl optionally substituted by one or more groups chosen, independently of one another, from the following groups: a halogen atom, a hydroxyl group, a (C₁-C₄)alkyl or a (C₁-C₄)alkoxy;
a heterocyclyl group, in particular pyridinyl or furanyl, optionally substituted by a hydroxyl, (C₁-C₄)alkyl or (C₁-C₄)alkoxy group; in the base, addition salt with an acid, hydrate or solvate state, in the form of enantiomers, of diastereoisomers, of rotamers, of atropoisomers or of their mixtures.

Mention may be made, among the compounds which are subject matters of the invention, of a third group of compounds of general formula (I) in which:
R represents:
   a hydrogen atom;
   a (C₁-C₄)alkyl;
R′ represents:
   a (C₁-C₄)alkyl optionally substituted by one or more groups chosen from:
      a hydroxyl group,
      a (C₁-C₄)alkoxy group,
      an aryloxy group, it being possible for said aryl to be substituted by one or more groups chosen from: a halogen atom, a (C₁-C₄)alkyl or a (C₁-C₄) alkoxy;
      an aryl group, it being possible for said aryl to be substituted by one or more groups chosen from: a halogen atom, a (C₁-C₄)alkyl or a (C₁-C₄) alkoxy;
      an NH(CO)R_{b} group, in which R_{b} represents an aryl group optionally substituted by one or more carboxylic acid groups;
      an —NR_{c}R_{d} group, in which R_{c} and R_{d} represent, independently of one another: a hydrogen atom or a (C₁-C₄)alkyl, or form, with the nitrogen atom which connects them, a heterocyclyl;
      a heterocyclyl group;
      a COORₑ group, in which Rₑ can represent a hydrogen or a (C₁-C₄)alkyl;
      a CONR_{c}R_{d} group;
   a COO(C₁-C₄)alkyl or a CONR_{c}R_{d};
Ar₁ represents:
a phenyl group optionally substituted by one or more groups chosen, independently of one another, from the following groups: a halogen atom, a (C₁-C₄)alkyl or a (C₁-C₄)alkoxy;
a pyridinyl group, said pyridinyl group optionally being substituted by a (C₁-C₄)alkyl;
T represents
a —CH₂— group;
Ar₂ represents:
a phenyl group optionally substituted by one or more groups chosen, independently of one another, from the following groups: a halogen atom, a (C₁-C₄)alkyl or a (C₁-C₄)alkoxy;
a pyridinyl group, optionally substituted by a halogen atom, a (C₁-C₄)alkyl or a (C₁-C₄)alkoxy;
Ar₃ represents
a phenyl group optionally substituted by one or more groups chosen, independently of one another, from the following groups: a halogen atom or a hydroxyl, (C₁-C₄)alkyl or (C₁-C₄)alkoxy group;
a pyridinyl or furanyl group, said groups optionally being substituted by a hydroxyl, (C₁-C₄)alkyl or (C₁-C₄)alkoxy group;
   in the base, addition salt with an acid, hydrate or solvate state, in the form of enantiomers, of diastereoisomers, of rotamers, of atropoisomers or of their mixtures.

When Ar₂ is an optionally substituted phenyl group, the T-Ar₂ bond, on the one hand, and Ar₂—N bond, on the other hand, are in the ortho position. In other words, the nitrogen atom and the substituent T are on two adjacent carbon atoms.

In the context of the invention:
a (C₁-C₄)alkyl is understood to mean a saturated, linear or branched, aliphatic group comprising from 1 to 4 carbon atoms, such as methyl, ethyl, propyl, isopropyl, butyl, isobutyl, sec-butyl or tert-butyl;
an optionally substituted (C₁-C₄)alkyl is understood to mean an alkyl group as defined above in which one or more hydrogen atoms have been replaced by a substituent; when several hydrogen atoms are replaced by fluorines, an optionally substituted (C₁-C₄)alkyl is understood to mean a perfluoroalkyl, such as —CF₃ or —C₂F₅;
a (C₁-C₄)alkoxy is understood to mean a (C₁-C₄)alkyl-O— radical where the (C₁-C₄)alkyl group is as defined above, for example methoxy, ethoxy, propoxy, isopropoxy, butoxy, isobutoxy, sec-butoxy or tert-butoxy;
a halogen atom is understood to mean a fluorine atom, a chlorine atom, a bromine atom or an iodine atom;
an aryl group is understood to mean a monocyclic or bicyclic aromatic group comprising between 6 and 10 carbon atoms, for example phenyl or naphthyl, it being possible for the aryl group optionally to be substituted by 1, 2, 3 or 4 substituents;
a heterocyclyl group is understood to mean a saturated, unsaturated or aromatic monocyclic group comprising between 4 and 7 atoms and comprising 1 or 2 heteroatoms chosen from nitrogen, oxygen or sulfur. Mention may be made, by way of example, of azetidinyl, piperidinyl, pyrrolidinyl, imidazolyl, pyridinyl, thiazolyl, thienyl, pyrimidinyl, furanyl or morpholinyl.

The compounds of general formula (I) can comprise one or more asymmetric carbons. They can thus exist in the form of enantiomers or of diastereoisomers. These enantiomers or diastereoisomers, and their mixtures, including racemic mixtures, come within the invention. Due to their structure, the compounds of general formula (I) can also exist in the form of rotamers. In the context of the invention, the term “rotamers” is understood to mean compounds which have identical expanded formulae but different fixed spatial conformations. These differences in the fixed spatial conformations of these compounds can confer different physicochemical properties on them and even, in some cases, different biological activities.

The compounds of general formula (I) can also exist in the form of atropoisomers. Atropoisomers are compounds with identical expanded formulae but which exhibit a specific spatial configuration resulting from a restricted rotation around a single bond due to high steric hindrance on either side of the single bond.

Atropoisomerism is independent of the presence of stereogenic components, such as an asymmetric carbon.

The compounds of formula (I) can exist in the state of bases or addition salts with acids. Such addition salts come within the invention.

These salts are advantageously prepared with pharmaceutically acceptable acids but the salts of other acids, for example of use in the purification or separation of the compounds of general formula (I), also come within the invention.

The compounds of general formula (I) can, in addition, occur in the form of hydrates or solvates, namely in the form of combinations or associations with one or more molecules of water or with a solvent. Such hydrates and solvates also come within the invention.

Another subject matter of the present invention is the process of the preparation of the compounds of general formula (I).

Thus, the compounds of general formula (I) can be prepared by the process illustrated in scheme 1. According to this scheme, the compounds of formula (I) can be obtained by condensation, in a basic medium, of an epoxide of formula (X) with the compounds of formula (II).

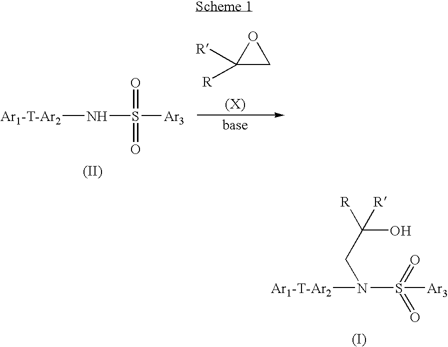

The base used can be a phosphazene. Mention may be made, by way of example, of 1-[N-(tert-butyl)-P,P-di(pyrrolidin-1-yl)phosphorimidoyl]pyrrolidine. This reaction is an adaptation of the process described by Karat L. D. et al., J. Appl. Chem. USSR, EN, 65, 6.2, 1992, 1130-1133.

The compounds of formula (II) are obtained beforehand according to scheme 2 by the sulfonylation of the compound of formula (III) with sulfonyl chlorides of formula (V) in the presence of a base chosen from tertiary amines, such as pyridine, according to the process described by Stauffer et al., Bioorg. Med. Chem., 2000, EN 8, 6, 1293-1316. Use may also be made, as tertiary amines, of triethylamine or diisopropyl-ethylamine or, in some cases, of a mixture of tertiary amines. When the compounds of formula (II) are obtained by reaction of the compounds of formula (IIIf) with the sulfonyl chlorides of formula (V), the ketone functional group of the compound obtained is subsequently reduced, according to methods known to a person skilled in the art, to result in the compounds of formula (II).

The compounds of formula (V) are commercially available or can be obtained by adaptation of the processes described, for example, by A. J. Prinsen et al., Recl. Trav. Chim. Pays-Bas, 1965, EN 84, 24.

In the compounds of formulae (III) and (V), Ar₁, Ar₂, Ar₃ and T are as defined in the formula (I).

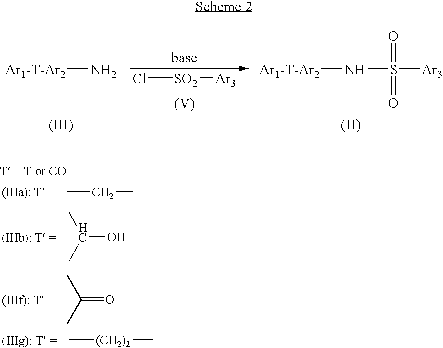

The compounds of formulae (IIIa), (IIIb) and (IIIf) are prepared according to schemes 3 to 5.

According to scheme 3, the 2-nitrobenzaldehyde derivatives of formula (VI) react with organometallic compounds of formula (VII), in which M represents an MgBr, MgI, ZnI or Li group, to result in the compounds of formula (VIII). The nitro functional group of the compounds of formula (VIII) is subsequently reduced by hydrogenation, for example under the action of metallic tin and concentrated hydrochloric acid in ethanol, to give the compounds of formula (IIIb). The derivatives of formula (IIIb) are reduced by the action of hydrides, for example with a mixture of triethylsilane and trifluoroacetic acid in dichloromethane, to result in the derivatives of formula (IIIa).

The organometallic compounds of formula (VII) are commercially available or are formed according to conventional processes described in the literature.

The nitrobenzaldehydes of formula (VI) are commercially available or can be prepared, for example, according to an adaptation of the process described by J. Kenneth Horner et al., J. Med. Chem., 1968, 11, 5, 946.

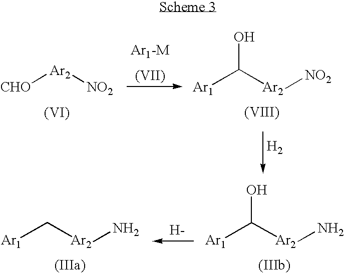

Other possibilities for synthesizing the compounds of general formulae (IIIb) and (IIIf) are presented in scheme 4.

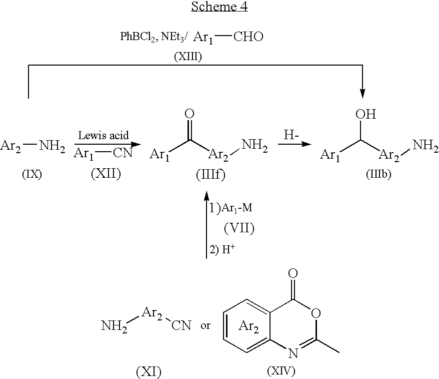

According to scheme 4, the anilines of formula (IX) are condensed with benzonitriles of formula (XII) in the presence of a Lewis acid, such as, for example, boron trichloride with aluminum trichloride or with gallium trichloride, to give the compounds of formula (IIIf), according to the process described by T. Sugasawa et al., J.A.C.S., 1978, 100, 4842. The compounds of formula (IIIf) can also be obtained by condensation of aminobenzonitriles (XI) with the organometallic derivatives (VII), followed by acid hydrolysis, according to the process described by R. Fryer et al., J. Heterocycl. Chem., 1991, EN 28, 7, 1661, or from the intermediate (XIV), according to an adaptation of the process described by D. Lednicer, J. Heterocyclic Chem., 1971, 903. The carbonyl functional group of the compounds (IIIf) can be reduced by the action of a hydride, for example sodium borohydride in ethanol, to result in the compounds of formula (IIIb).

Another method for the preparation of the compounds of formula (IIIb) consists in condensing anilines of formula (IX) with benzaldehyde derivatives of formula (XIII) in the presence of phenyldichloroborane and triethylamine, according to the process described by T. Toyoda et al., Tet. Lett., 1980, 21, 173.

It should be noted that the compounds of formula (IIIf) can result, for example under the action of triethylsilane and trifluoroacetic acid, in the compounds of formula (IIIa).

Another possibility for synthesizing the compounds of general formula (IIIa) in which Ar₁ represents a heteroaryl is presented in scheme 5.

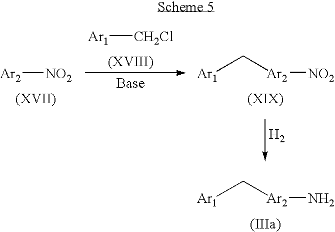

The nitrophenyls of formula (XVII) are condensed with aromatic chloromethylheterocyclyls of formula (XVIII) in the presence of a base, for example potassium tert-butoxide, to result in the derivatives (XIX), according to the process described by Florio S. et al., Eur. J. Org. Chem., 2004, 2118, which derivatives are reduced, for example by the action of metallic tin in the presence of 12M hydrochloric acid, to result in the derivatives of formula (IIIa).

The compounds of formula (IIIg) are prepared according to scheme 6. The nitrobenzaldehydes (VI), by condensation with the derivatives (XV) according to a Wittig reaction, result in the compounds (XVI). These derivatives are reduced, for example by catalytic hydrogenation with palladium, to give the compounds of formula (IIIg).

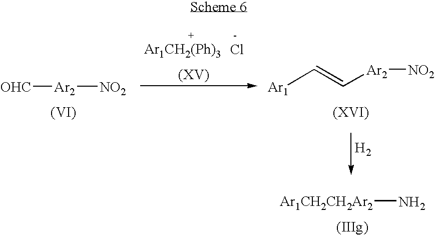

In all the schemes and for all the compounds of formulae (II) to (XIX), the meanings of Ar₁, T, Ar₂, Ar₃ and R₁ are as defined for the compounds of general formula (I).

In schemes 1 to 6, the starting compounds and the reactants, when their method of preparation is not described, are commercially available or are described in the literature or else can be prepared by methods which are described therein or which are known to a person skilled in the art.

When a compound comprises a reactive functional group, for example a hydroxyl group, it may require prior protection before reaction. A person skilled in the art can determine the need for prior protection.

The compounds of formulae (II) to (XIX) are of use as synthetic intermediates in the preparation of the compounds of general formula (I) and form an integral part of the present invention.

### EXAMPLES

The following examples describe the preparation of the compounds in accordance with the invention. These examples are nonlimiting and serve only to illustrate the invention.

The numbers of the compounds in examples refer to those given in the table. The elemental microanalyses, the mass spectra and the NMR spectra confirm the structures of the compounds obtained.

The conditions for analysis by liquid chromatography coupled to a mass spectrometry LC/MS are as follows:
for the liquid chromatography part: Column symmetry C18 (2.1×50 mm) 3-5 μm. Eluent A=H₂O+0.005% of TFA, pH=3.14; Eluent B=CH₃CN+0.005% of TFA, with a gradient from 100% of A to 90% of B in 10 minutes, then 5 minutes at 90% of B,
for the mass spectrometry part: positive electrospray ionization mode.

When the ¹H NMR spectrum demonstrates rotamers, only the interpretation corresponding to the predominant rotamer is described.

### Example 1

### N-[2-(2,6-Difluorobenzyl)-6-methoxyphenyl]-N-(2,3-dihydroxypropyl)-3,4-dimethoxybenzenesulfonamide (compound No. 1)

74 mg of oxiran-2-ylmethanol are added to 224 mg of N-[2-(2,6-difluorobenzyl)-6-methoxyphenyl]-3,4-dimethoxybenzenesulfonamide and 234 mg of 1-[N-(tert-butyl)-P,P-di(pyrrolidin-1-yl)phosphorimidoyl]-pyrrolidine in solution in 5 ml of tetrahydrofuran. After 72 hours at ambient temperature, the medium is directly chromatographed on a column of silica gel, elution being carried out with a water+0.05% of trifluoroacetic acid/acetonitrile+0.05% of trifluoroacetic acid mixture, in order to obtain 102 mg of the expected product.

¹H NMR 5 in ppm (d₆-DMSO): 3.20 (3H, 2-OMe), 3.41 (2H, 14-H₂), 3.80 (1H, 13-H), 3.72 (3H, 17-OMe), 3.84/3.03 (2H, 12-H₂), 3.86 (3H, 18-OMe), 4.50/4.39 (2H, 7-H₂), 4.60 (1H, 14-OH), 4.62 (1H, 13-OH), 6.16 (1H, 5-H), 6.81 (1H, 3-H), 7.03 (1H, 16-H), 7.14 (1H, 10/10′-H), 7.14 (1H, 19-H), 7.15 (1H, 4-H), 7.25 (1H, 20-H), 7.42 (1H, 11-H).

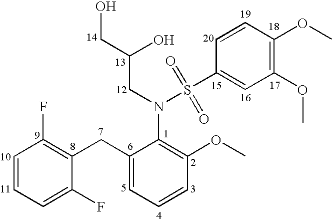

In the following table:
MH⁺ represents the mass peak of the ionized product
the retention time is expressed in minutes
n.d. means “not determined”
Me represents a methyl group
(R) and (S) represents indicates the asymmetry of the carbon

TABLE 1

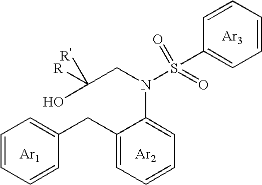

| Com- | Nature and position of the substituents | | | | | MH⁺/ |
|---|---|---|---|---|---|---|
| pound | | | on | | | retention |
| No. | R | on Ar₁ | Ar₂ | R′ | on Ar₃ | time |
| 2 | H | 2,6-diF | H | CH₂—OCH₃ | 3,4-diOMe | 538/1.79 |
| 3 | H | 2,6-diF | H | 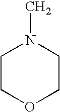 | 3,4-diOMe | 593/1.39 |
| 4 | H | 2,6-diF | H | COOCH₃ (R) | 3,4-diOMe | 552/1.79 |
| 5 | H | 2,6-diF | H | COOCH₃ (S) | 3,4-diOMe | 552/1.78 |
| 6 | H | 2,6-diF | H | 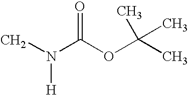 | 3,4-diOMe | 623/1.93 |
| 7 | H | 2,6-diF | H | CH₃ | 3,4-diOMe | 508/1.58 |
| 8 | CH₃ | 2,6-diF | H | CH₃ | 3,4-diOMe | 522/1.86 |
| 9 | H | 2,6-diF | H | C(O)NH₂ | 3,4-diOMe | 537/1.59 |
| 10 | H | 2,6-diF | H | CF₃ | 3,4-diOMe | 562/1.97 |
| 11 | H | 2,6-diF | H | 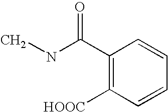 | 3,4-diOMe | 669/2.02 |
| 12 | CH₃ | 2,6-diF | H | COOCH₃ | 3,4-diOMe | 566/1.82 |
| 13 | H | 2,6-diF | H | 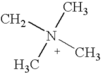 | 3,4-diOMe | 565/1.47 |
| 14* | H | 2,6-diF | H | CH₂—NH₂ | 3,4-diOMe | 523/1.45 |
| 15 | CH₃ | 2,6-diF | H | COOH | 3,4-diOMe | 552/1.67 |
| 16 | H | 2,6-diF | H | COOH (R) | 3,4-diOMe | 538/1.63 |
| 17 | H | 2,6-diF | H | COOH (S) | 3,4-diOMe | 536/1.33 |

*trifluoroacetic acid salt

### Pharmacological Testing

The compounds of the invention have formed the subject of pharmacological studies which have shown their advantage as therapeutically active substances.

They have in particular been tested with regard to their effects. More particularly, the affinity of the compounds of the invention for the orexin 2 receptors was determined in a test of in vitro binding according to the technique described below. This method consists in studying the displacement of radioiodinated orexin A bound to human orexin 2 receptors expressed in CHO cells. The test is carried out on membranes in an incubation buffer of 50 mm Hepes, 1 mM MgCl₂, 25 mm CaCl₂, 0.025% NaN₃, 1% bovine serum albumin (BSA) and 100 pM of ligand for 30 minutes at 25° C. The reaction is halted by filtering and washing on a Whatman GF/C filter. The nonspecific binding is measured in the presence of 10⁻⁶ M of human orexin B. The IC₅₀ (concentration which inhibits the binding of the radioiodinated orexin A to its receptors by 50%) values are low, less than 300 nM, in particular less than 100 nM and more particularly less than 30 nM.

The affinity of some compounds according to the invention for the oxerin receptor is illustrated in the following table.

| | IC₅₀ OX 2 |
|---|---|
| Compound No. | (nM) |
| 7 | 37 |
| 11 | 15 |

The biological results show that the compounds according to the invention are indeed antagonists of the orexin 2 receptors.

Thus, the compounds of the present invention, as antagonists of the orexin 2 receptors, can be used in the prophylaxis and treatment of any disease involving a dysfunctioning related to these receptors.

The compounds of the invention can be used in the preparation of a medicament intended for the prophylaxis or treatment of any disease involving a dysfunctioning related to the orexin 2 receptor and more particularly in the prophylaxis or treatment of pathologies in which an orexin 2 receptor antagonist provides a therapeutic benefit. Such pathologies are, for example, obesity, appetite or taste disturbances, including cachexia, anorexia or bulimia (Smart et al., Eur. J. Pharmacol., 2002, 440, 2-3, 199-212), diabetes (Ouedraogo et al., Diabetes, 2002, 52, 111-117), metabolic syndromes (Sakurai, Curr. Opin. Nutr. Metab. Care, 2003, 6, 353-360), vomiting and nausea (U.S. Pat. No. 6,506,774), depression and anxiety (Salomon et al., Biol. Psychiatry, 2003, 54, 96-104; Jaszberenyi et al., J. Neuroendocrinol., 2000, 12, 1174-1178), addictions (Georgescu et al., J. Neurosci., 2003, 23, 8, 3106-3111; Kane et al., Endocrinology, 2000, 141, 10, 3623-3629), mood and behavioral disorders, schizophrenia (Nishino et al., Psychiatry Res., 2002, 110, 1-7), sleep disorders (Sakurai, Neuroreport, 2002, 13, 8, 987-995), restless legs syndrome (Allen et al., Neurology, 2002, 59, 4, 639-641), learning and memory disorders (van den Pol et al., 2002, J. Physiol., 541(1), 169-185; Jaeger et al., Peptides, 2003, 23, 1683-1688; Telegdy and Adamik, Regul. Pept., 2002, 104, 105-110), sexual and psychosexual dysfunctions (Gulia et al., Neuroscience, 2003, 116, 921-923), pain, visceral or neuropathic pain, hyperalgesia, allodynia (U.S. Pat. No. 6,506,774; Suyama et al., In vivo, 2004, 18, 2, 119-123), digestive disorders (Takakashi et al., Biochem. Biophy. Res. Comm., 1999, 254, 623-627; Matsuo et al., Eur. J. Pharmacol., 2002, 105-109), irritable bowel syndrome (U.S. Pat. No. 6,506,774), neuronal degeneration (van den Pol, Neuron, 2000, 27, 415-418), ischemic or hemorrhagic strokes (Irving et al., Neurosci. Lett., 2002, 324, 53-56), Cushing's disease, Guillain-Barré syndrome (Kanbayashi et al., Psychiatry Clin. Neurosci., 2002, 56, 3, 273-274), myotonic dystrophy (Martinez-Rodriguez et al., Sleep, 2003, 26, 3, 287-290), urinary incontinence (Blackstone et al., AGS Annual Meeting, poster P491, 2002), hyperthyroidism (Malendowicz et al., Biomed. Res., 2001, 22, 5, 229-233), disorders of pituitary function (Voisin et al., Cell. Mol. Life. Sci., 2003, 60, 72-78), hypertension or hypotension (Samson et al., Brain Res., 1999, 831, 1-2, 248-253).

The use of the compounds according to the invention in the preparation of a medicament intended to prevent or treat the abovementioned pathologies forms an integral part of the invention.

Another subject matter of the invention is medicaments which comprise a compound of formula (I). These medicaments are employed therapeutically, in particular in the prophylaxis or treatment of the abovementioned pathologies.

According to another of its aspects, the present invention relates to pharmaceutical compositions including, as active principle, at least one compound according to the invention. These pharmaceutical compositions comprise an effective dose of a compound according to the invention and optionally one or more pharmaceutically acceptable excipients.

Said excipients are chosen, depending on the pharmaceutical form and the method of administration desired, from the usual excipients which are known to a person skilled in the art.

In the pharmaceutical compositions of the present invention for oral, sublingual, subcutaneous, intramuscular, intravenous, topical, local, intratracheal, intranasal, transdermic or rectal administration, the active principle of formula (I) above or its optional salt, solvate or hydrate can be administered in unit administration form, as a mixture with conventional pharmaceutical excipients, to animals and human beings for the prophylaxis or treatment of the above disorders or diseases.

Appropriate unit administration forms comprise oral forms, such as tablets, soft or hard gelatin capsules, powders, granules, chewing gums and oral solutions or suspensions, forms for sublingual, buccal, intratracheal, intraocular or intranasal administration or for administration by inhalation, forms for subcutaneous, intramuscular or intravenous administration and forms for rectal or vaginal administration. For topical application, the compounds according to the invention can be used in creams, ointments or lotions.

For example, when a solid composition in the form of tablets is prepared, the main active ingredient is mixed with a pharmaceutical excipient, such as gelatin, starch, lactose, magnesium stearate, talc, gum arabic or the like. The tablets can be coated with sucrose, with a cellulose derivative or with other materials. The tablets can be produced by different techniques: direct tableting, dry granulation, wet granulation or hot melt.

In order to obtain the desired prophylactic or therapeutic effect, the dose of active principle can vary between 0.1 mg and 200 mg per kg of body weight and per day. Although these dosages are examples of an average situation, there may be specific cases where higher or lower dosages are appropriate; such dosages also come within the invention. According to the usual practice, the dosage appropriate to each patient is determined by the physician according to the method of administration and the weight and the response of said patient.

Each unit dose can comprise from 0.1 to 1000 mg, preferably from 0.1 to 500 mg, of active principle, in combination with one or more pharmaceutical excipients. This unit dose can be administered 1 to 5 times daily, so as to administer a daily dose of 0.5 to 5000 mg, preferably of 0.5 to 2500 mg.

The present invention, according to another of its aspects, also relates to a method for preventing or treating the pathologies indicated above which comprises the administration of a compound according to the invention, of a pharmaceutically acceptable salt of said compound, of a solvate of said compound or of a hydrate of said compound.

We claim:

1. A compound of formula (I) 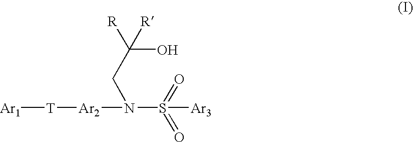
wherein:
R is a hydrogen atom or a (C₁-C₄)alkyl group;
R′ is a (C₁-C₄)alkyl group optionally substituted by one or more groups chosen from:
a hydroxyl group,
a halogen group,
a (C₁-C₄)alkoxy group,
an aryloxy group, optionally substituted by one or more groups chosen from a halogen atom, or a (C₁-C₄)alkyl or (C₁-C₄)alkoxy group,
an aryl group, optionally substituted by one or more groups chosen from a halogen atom, or a (C₁-C₄)alkyl or (C₁-C₄)alkoxy group,
an —NH(COO)Rₐ group, wherein Rₐ is a (C₁-C₄) alkyl group;
an —NH(CO)R_{b} group, wherein R_{b} is a (C₁-C₄)alkyl or an aryl group, and the said aryl group is optionally substituted by one or more groups chosen from a halogen atom, or a (C₁-C₄)alkyl, (C₁-C₄)alkoxy or carboxylic acid group,
an —NR_{c}R_{d} or —CONR_{c}R_{d} group, wherein R_{c} and R_{d} are independently of one another a hydrogen atom or a (C₁-C₄)alkyl group, or R_{c} and R_{d} taken together with the nitrogen atom to which they are attached form a heterocyclyl group,
an —NR_{f}R_{g}Rₕ ammonium group, wherein R_{f}, R_{g} and Rₕ are a (C₁-C₄)alkyl group,
a heterocyclyl group, or
a —COORₑ group, wherein Rₑ is a hydrogen atom or a (C₁-C₄)alkyl group, or
a —COOH, —COO(C₁-C₄)alkyl, or —CONR_{c}R_{d} group
Ar₁ is an aryl group optionally substituted by one or more groups chosen, independently of one another, from a halogen atom, cyano, or a (C₁-C₄)alkyl, fluoro(C₁-C₄)alkyl or (C₁-C₄)alkoxy group, or
a heterocyclyl group optionally substituted by a halogen atom, or a (C₁-C₄)alkyl or (C₁-C₄) alkoxy group;
T is a —(CH₂)ₙ— group, wherein n=1 or 2, or
a group: 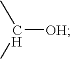
Ar₂ is an aryl group optionally substituted by one or more groups chosen, independently of one another, from a halogen atom, or a (C₁-C₄)alkyl, (C₁-C₄)alkoxy, fluoro(C₁-C₄)alkyl or fluoro(C₁-C₄)alkoxy group, or
a heterocyclyl group optionally substituted by a halogen atom, or a (C₁-C₄)alkyl or (C₁-C₄) alkoxy group; and
Ar₃ is an aryl group optionally substituted by one or more groups chosen, independently of one another, from a halogen atom, a hydroxyl group, (C₁-C₄)alkyl or (C₁-C₄)alkoxy group, or
a heterocyclyl group optionally substituted by a hydroxyl group, (C₁-C₄)alkyl, (C₁-C₄)alkoxy, fluoro(C₁-C₄)alkyl or fluoro(C₁-C₄)alkoxy group;
or an addition salt with an acid thereof, or a hydrate or solvate thereof.

2. The compound according to claim 1, wherein:
R′ is a (C₁-C₄)alkyl group optionally substituted by one or more groups chosen from:
a hydroxyl group,
a halogen group,
a (C₁-C₄)alkoxy group,
an aryloxy group, optionally substituted by one or more groups chosen from a halogen atom, or a (C₁-C₄)alkyl or (C₁-C₄)alkoxy group,
an aryl group, optionally substituted by one or more groups chosen from a halogen atom, or a (C₁-C₄)alkyl or (C₁-C₄)alkoxy group,
an —NH(COO)Rₐ group, wherein Rₐ is a (C₁-C₄) alkyl group,
an NH(CO)R_{b} group, wherein R_{b} is an aryl group optionally substituted by one or more groups chosen from a halogen atom, or a (C₁-C₄) alkyl, (C₁-C₄) alkoxy or carboxylic acid group,
an —NR_{c}R_{d} or —CONR_{c}R_{d} group, wherein R_{c} and R_{d} are independently of one another a hydrogen atom or a (C₁-C₄)alkyl group, or R_{c} and R_{d} taken together with the nitrogen atom to which they are attached form a heterocyclyl group,
an —NR_{f}R_{g}Rₕ ammonium group, wherein R_{f}, R_{g} and Rₕ are a (C₁-C₄)alkyl group,
a heterocyclyl group, or
a COORₑ group, wherein Rₑ is a hydrogen atom or a (C₁-C₄)alkyl group, or
a —COO(C₁-C₄) alkyl or —CONR_{c}R_{d} group;
Ar₁ is phenyl or naphthyl, each of which is optionally substituted by one or more groups chosen, independently of one another, from a halogen atom, or a (C₁-C₄)alkyl or (C₁-C₄)alkoxy group, or
pyridinyl or pyrimidinyl, each of which is optionally substituted by a halogen atom, (C₁-C₄)alkyl or (C₁-C₄)alkoxy;
T is a —CH₂— group;
Ar₂ is phenyl, or naphthyl, each of which is optionally substituted by one or more groups chosen, independently of one another, from a halogen atom, or a (C₁-C₄)alkyl or (C₁-C₄)alkoxy group, or
pyridinyl, optionally substituted by a halogen atom, or a (C₁-C₄)alkyl or (C₁-C₄)alkoxy group; and
Ar₃ is phenyl or naphthyl, each of which is optionally substituted by one or more groups chosen, independently of one another, from a halogen atom, or a hydroxyl, (C₁-C₄)alkyl or (C₁-C₄)alkoxy group, or
pyridinyl or furanyl, each of which is optionally substituted by a hydroxyl, (C₁-C₄) alkyl or (C₁-C₄) alkoxy group;
or an addition salt with an acid thereof, or a hydrate or solvate thereof.

3. The compound according to claim 1:
R′ is a (C₁-C₄)alkyl group optionally substituted by one or more groups chosen from:
a hydroxyl group,
a (C₁-C₄)alkoxy group,
an aryloxy group, optionally substituted by one or more groups chosen from a halogen atom, (C₁-C₄)alkyl or (C₁-C₄)alkoxy,
an aryl group, optionally substituted by one or more groups chosen from a halogen atom, (C₁-C₄)alkyl or (C₁-C₄)alkoxy,
an NH(CO)R_{b} group, wherein R_{b} is an aryl group optionally substituted by one or more carboxylic acid groups,
an —NR_{c}R_{d} or —CONR_{c}R_{d} group, in which R_{c} and R_{d} are independently of one another: a hydrogen atom or (C₁-C₄)alkyl, or R_{c} and R_{d} taken together with the nitrogen atom to which they are attached form a heterocyclyl,
a heterocyclyl group, or
a COORₑ group, wherein Rₑ is a hydrogen atom or a (C₁-C₄)alkyl group, or
a —COO(C₁-C₄)alkyl, or —CONR_{c}R_{d} group;
Ar₁ is phenyl optionally substituted by one or more groups chosen, independently of one another, from a halogen atom, or a (C₁-C₄)alkyl or (C₁-C₄)alkoxy group, or
Pyridinyl optionally substituted by a (C₁-C₄) alkyl group;
T is a —CH₂— group;
Ar₂ is phenyl optionally substituted by one or more groups chosen, independently of one another, from a halogen atom, (a C₁-C₄)alkyl or (C₁-C₄)alkoxy group, or
pyridinyl optionally substituted by a halogen atom, or a (C₁-C₄)alkyl or (C₁-C₄)alkoxy group; and
Ar₃ is phenyl optionally substituted by one or more groups chosen, independently of one another, from a halogen atom, or a hydroxyl, (C₁-C₄)alkyl or (C₁-C₄)alkoxy group, or
pyridinyl or furanyl, each of which is optionally substituted by a hydroxyl, (C₁-C₄)alkyl or (C₁-C₄)alkoxy group;
or an addition salt with an acid thereof, or a hydrate or solvate thereof.

4. A process for preparing the compound according to claim 1, comprising condensing an epoxide of formula (X), in a basic medium, with a compound of formula (II): 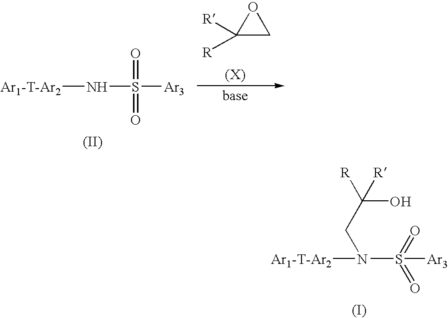
Wherein Ar₁, Ar₂, Ar₃, T, R and R′ are as defined in claim 1.

5. The process according to claim 4, wherein the compound of formula (II) is prepared by sulfonylating a compound of formula (III) with a sulfonyl chloride of formula (V) in the presence of a base:

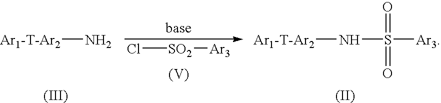

6. A method for treating obesity, appetite or taste disturbance, cachexia, anorexia, bulimia, diabetes, metabolic syndrome, vomiting, nausea, depression, anxiety, addiction, mood or behavioral disorder, schizophrenia, sleep disorder, restless leg syndrome, learning or memory disorder, sexual or psychosexual dysfunction, pain, visceral or neuropathic pain, hyperalgesia, allodynia, digestive disorder, irritable bowel syndrome, neuronal degeneration, ischemic or hemorrhagic strokes, Cushing's disease, Guillain-Barré syndrome, myotonic dystrophy, urinary incontinence, hyperthyroidism, disorder of pituitary function, hypertension or hypotension, in a patient in need thereof, comprising administering to the patient a pharmaceutically effective amount of the compound according to claim 1, or an addition salt with an acid thereof, or a hydrate or solvate thereof.

7. A method for treating obesity, appetite or taste disturbance, cachexia, anorexia, bulimia, diabetes, metabolic syndrome, vomiting, nausea, depression, anxiety, addiction, mood or behavioral disorder, schizophrenia, sleep disorder, restless leg syndrome, learning or memory disorder, sexual or psychosexual dysfunction, pain, visceral or neuropathic pain, hyperalgesia, allodynia, digestive disorder, irritable bowel syndrome, neuronal degeneration, ischemic or hemorrhagic strokes, Cushing's disease, Guillain-Barré syndrome, myotonic dystrophy, urinary incontinence, hyperthyroidism, disorder of pituitary function, hypertension or hypotension, in a patient in need thereof, comprising administering to the patient a pharmaceutically effective amount of the compound according to claim 2, or an addition salt with an acid thereof, or a hydrate or solvate thereof.

8. A method for treating obesity, appetite or taste disturbance, cachexia, anorexia, bulimia, diabetes, metabolic syndrome, vomiting, nausea, depression, anxiety, addiction, mood or behavioral disorder, schizophrenia, sleep disorder, restless leg syndrome, learning or memory disorder, sexual or psychosexual dysfunction, pain, visceral or neuropathic pain, hyperalgesia, allodynia, digestive disorder, irritable bowel syndrome, neuronal degeneration, ischemic or hemorrhagic strokes, Cushing's disease, Guillain-Barré syndrome, myotonic dystrophy, urinary incontinence, hyperthyroidism, disorder of pituitary function, hypertension or hypotension, in a patient in need thereof, comprising administering to the patient a pharmaceutically effective amount of the compound according to claim 3, or an addition salt with an acid thereof, or a hydrate or solvate thereof.

9. A pharmaceutical composition comprising the compound according to claim 1, or an addition salt with an acid thereof, or a hydrate or solvate thereof, in combination with at least one pharmaceutically acceptable excipient.

10. A pharmaceutical composition comprising the compound according to claim 2, or an addition salt with an acid thereof, or a hydrate or solvate thereof, in combination with at least one pharmaceutically acceptable excipient.

11. A pharmaceutical composition comprising the compound according to claim 3, or an addition salt with an acid thereof, or a hydrate or solvate thereof, in combination with at least one pharmaceutically acceptable excipient.
